(19) United States
(12) Patent Application Publication
(10) Pub. No.: US 2014/0030769 A1
(43) Pub. Date: Jan. 30, 2014

(54) FREE ENZYME AND CELLULOSOME PREPARATIONS FOR CELLULOSE HYDROLYSIS

(71) Applicant: Alliance for Sustainable Energy, LLC, Golden, CO (US)

(72) Inventors: Michael RESCH, Golden, CO (US); John O. BAKER, Golden, CO (US); Xu QI, Golden, CO (US); William S. ADNEY, Golden, CO (US); Steven R. DECKER, Golden, CO (US); Michael E. HIMMEL, Golden, CO (US); Bryon DONOHOE, Golden, CO (US)

(73) Assignee: Alliance for Sustainable Energy, LLC, Golden, CO (US)

(21) Appl. No.: 13/953,220

(22) Filed: Jul. 29, 2013

Related U.S. Application Data
(60) Provisional application No. 61/676,401, filed on Jul. 27, 2012.

(51) Int. Cl. C12N 9/42; C12P 7/14; C12P 19/14

(52) U.S. Cl. CPC C12N 9/2437; C12P 19/14; C12P 7/14
USPC 435/99; 435/209; 435/162

(57) ABSTRACT

Disclosed herein are combinations of free fungal enzymes and cellulosomes useful for the hydrolysis of cellulose and the conversion of biomass. Methods of degrading cellulose and biomass using the combinations are also disclosed.

### CROSS-REFERENCE TO RELATED APPLICATIONS

This application claims priority to U.S. Provisional Application No. 61/676,401, filed Jul. 27, 2012, the contents of which are incorporated by reference in their entirety.

### CONTRACTUAL ORIGIN

The United States Government has rights in this invention under Contract No. DE-AC36-08G028308 between the United States Department of Energy and Alliance for Sustainable Energy, LLC, the Manager and Operator of the National Renewable Energy Laboratory.

### BACKGROUND

Plant cell walls represent a vast, renewable carbon source in the biosphere. Biofuels derived from plant cell wall material is a promising renewable energy technology in part because of the large amount and low cost of the biomass feedstock. Efficient action of cellulases to release fermentable sugars from biomass cellulose is an important step in making this conversion economically viable.

Nature has evolved multiple enzymatic strategies for the degradation of plant cell wall polysaccharides that are central to carbon and nitrogen flux in the biosphere and an integral part of renewable biofuels development. Many biomass-degrading organisms secrete cocktails of individual enzymes with one or a few catalytic domains per enzyme, whereas some bacteria synthesize large multi-enzyme complexes, termed cellulosomes, which may contain 50-60 catalytic units per complex. Both enzyme systems employ similar catalytic chemistry, but the physical mechanisms by which these enzyme systems degrade polysaccharides have not been compared directly.

These enzymatic strategies largely rely on glycoside hydrolases, oxidative enzymes, and other accessory proteins. Secreted free enzyme cocktails typically contain various proteins that diffuse independently of one another and, via different substrate specificities, work together to degrade biomass. These free enzymes range from systems in which the enzymes contain one catalytic unit to systems in which there may be several catalytic units per protein. In particular, the fungus Hypocrea jecorina (formally Trichoderma reesei) secretes a potent cocktail of free carbohydrate-active enzymes to degrade cellulose and hemicellulose. The T. reesei enzyme cocktail and related systems typically secrete enzymes with only one catalytic unit per protein.

Sorting through the vast array of bacterial and fungal enzymes to determine the optimal combination of activities for cellulose degradation is a continuing challenge to the biofuels industry. Discovering enzyme cocktails that work together to increase the efficiency of sugar release from biomass sources is essential to making biofuels economically competitive with petroleum-based fuels.

The foregoing examples of the related art and limitations related therewith are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods that are meant to be exemplary and illustrative, not limiting in scope. In various embodiments, one or more of the above-described problems have been reduced or eliminated, while other embodiments are directed to other improvements.

Exemplary embodiments provide methods for degrading cellulose or lignocellulosic biomass by contacting a cellulose containing material or lignocellulosic biomass with an enzyme cocktail comprising at least one fungal cellulase and at least one cellulosome complex.

In certain embodiments, the cellulosome complex is a high molecular weight (HMW) cellulosome complex. In some embodiments, the cellulosome complex is from a bacterium of the genus Clostridium, such as the bacterium C. thermocellum.

In some embodiments, the fungal cellulase comprises a Family 7 cellobiohydrolase such as Cel7A. In certain embodiments, the Family 7 cellobiohydrolase is from a fungus of the genus Hypocrea, such as the fungus H. jecorina.

In further embodiments, the enzyme cocktail further comprises a β-glucosidase, a hemicellulase, or an oxidoreductase. In some embodiments, the cellulase comprises a commercial enzyme preparation such as CTec2.

In certain embodiments, the contacting a cellulose containing material or lignocellulosic biomass is carried out at a temperature of between 50-60° C. or at 50° C.

Also provided are enzyme cocktails comprising at least one HMW cellulosome complex and a Family 7 cellobiohydrolase.

Further provided are methods for producing a biofuel from lignocellulosic biomass by contacting the lignocellulosic biomass with an enzyme cocktail described herein and converting the sugars to a biofuel by fermentation.

In addition to the exemplary aspects and embodiments described above, further aspects and embodiments will become apparent by reference to the drawings and by study of the following descriptions.

### BRIEF DESCRIPTION OF THE DRAWINGS

Exemplary embodiments are illustrated in referenced figures of the drawings. It is intended that the embodiments and figures disclosed herein are to be considered illustrative rather than limiting.

FIG. 1 shows a comparison of cellulosomes and free enzymes in the digestion of cellulose and biomass substrates Avicel (A), Whatman filter paper (B), dilute acid pretreated switchgrass (C) and dilute acid pretreated poplar (D).

FIG. 2 shows TEM micrographs of Avicel particles digested with free enzymes or cellulosomes. Scale bars A-C, F, G=200 nm, D, H=500 nm, E=100 nm.

FIG. 3 shows TEM micrographs of immuno-labeled dilute acid pretreated switchgrass samples digested with free enzymes (A, A′) or cellulosomes (B, B′) for 24 hours. Scale bars=0.5 μm.

FIG. 4 illustrates the synergistic effects of free enzymes and cellulosomes on Avicel examined by activity assays and TEM imaging. Scale bars=500 nm.

FIG. 5 shows an illustration of the mechanisms by which free enzymes (left) and cellulosomes (right) differ in their action on cellulose microfibril bundles.

FIG. 6A illustrates size exclusion chromatography (SEC) separation and pooling of fractions containing the HMW cellulosomes. FIG. 6B shows results from native PAGE analysis used to identify the fractions that contained HMW cellulosomes.

FIG. 7 illustrates enhanced cellulase activity of chromatographically-selected cellulosome fraction.

FIG. 8 shows an optimization of cellulosome enzymatic activity conditions on Avicel as a function of aerobic or anaerobic conditions, β-glucosidase presence, and the presence of chemical protectants.

FIG. 9 shows a comparison of cellulosome and free enzyme (CTec2) hydrolytic activity on phosphoric acid swollen cellulose (PASC).

FIG. 10 shows the effect of adding hemicellulase enzymes to the cell free cellulosome on pretreated (A) or untreated (B) switchgrass enzymatic digestions.

FIG. 11 shows TEM micrographs of immuno-labeled Avicel PH101 digested with CTec2 for 120 hours (A, B) or HMW cellulosomes for 24 hours (C, D) to achieve a cellulose conversion of about 65% in each case. Scale bars=200 nm.

FIG. 12 shows higher magnification TEM micrographs of dilute acid pretreated switchgrass samples (A, B) and enzymatic digestions of pretreated switchgrass (A′, B′). Scale bar=2 μm.

### DETAILED DESCRIPTION

Disclosed herein are enzyme combinations useful for the hydrolysis of cellulose and the conversion of biomass. In particular, combinations of free fungal enzymes with cellulosomes show synergistic activities on cellulose-containing substrates. Methods of degrading cellulose and biomass using enzyme and cellulosome combinations and cocktails of enzymes and cellulosomes are also disclosed.

In bacterial and fungal free enzyme systems, distinct families of processive cellulases have evolved to hydrolyze cellulose from either the reducing or non-reducing end. Processive cellulases are complemented by the presence of non-processive cellulases and oxidative enzymes that cleave cellulose chains to expose free ends for attachment of processive enzymes. Co-secreted hemicellulase enzymes target the variety of glycosidic linkages in hemicellulose and work in conjunction with various esterases, pectinases, and other accessory enzymes. Generally, once exposed to biomass, free enzymes can diffuse throughout the cell wall matrix to degrade their target cell wall components.

An alternative degradation paradigm has evolved in certain bacteria in which multiple biomass-degrading enzymes are physically connected via an anchoring protein scaffold. This macromolecular enzyme complex, termed the cellulosome, can be found in certain bacteria, such as the anaerobic bacterium Clostridium thermocellum. In contrast to cellulase cocktails (such as those from T. reesei) in which single catalytic units exist on each protein, the cellulosome paradigm represents the opposite end of known biomass degradation strategies wherein many catalytic units are physically linked together to form a large mega-dalton (MDa) complex with presumably limited intra-cell wall diffusion capability.

Cellulosomes incorporate processive and non-processive cellulases, hemicellulases, and other carbohydrate-active enzymes onto large non-catalytic proteins known as scaffoldins. The predominant interaction that enables enzyme binding to scaffoldins is the tight, highly-specific noncovalent attachment of the dockerin domains of cellulosomal enzymes to multiple cohesin domains that are distributed along each scaffoldin peptide. Primary scaffoldins can bind up to nine enzymes via Type I cohesins, which are complementary to the Type I dockerins on individual enzymes. Primary scaffoldins also typically contain a carbohydrate binding module (CBM) and a Type II dockerin domain.

Up to seven primary scaffoldins, along with their associated enzymes, can attach through Type II dockerins to Type II cohesins of secondary scaffoldins to form large, multi-enzyme complexes incorporating up to 50 to 60 catalytic units per cellulosome complex. These secondary scaffoldins can in turn adhere to bacterial cell surfaces or exist freely in solution. This complexed enzyme architecture can facilitate diverse assemblies of enzymes and CBMs with aggregate molecular masses up to 10 MDa. The proximity of CBMs and carbohydrate-active enzymes with multiple binding preferences and substrate specificities, respectively, bound to long, flexible scaffoldins has long been hypothesized to impart “plasticity” (or variable quaternary structure) to the cellulosome, which in turn has been hypothesized to yield enhanced activity.

The organization of catalytic units and CBMs in the cellulosome is distinct from the free enzymes and, as described here, this structural difference translates into different enzymatic performance on different substrates. Typically, free fungal enzymes are more active on thermochemically treated biomass than are cellulosomes, while the cellulosomes are better at the digestion of pure cellulose. Transmission electron microscopy (TEM) imaging of partially digested substrates reveals a mechanism of biomass degradation by cellulosomes that is different from the well-known fibril sharpening, ablative mechanism of free cellulases. In contrast to the shape-alteration produced by the free enzymes, cellulosomes splay open one of the ends of cellulose bundles, increasing the separation distance between individual cellulose microfibrils.

Herein we disclose that free-enzyme cocktails such as those expressed by Hypocrea jecorina and cellulosomes such as those from Clostridium thermocellum, when combined, display synergistic enzyme activity. TEM images suggest very different mechanisms of cellulose deconstruction by free enzymes and cellulosomes. Specifically, the free enzymes employ an ablative mechanism, whereas cellulosomes physically separate individual cellulose microfibrils from larger particles for enhanced access to the cellulose surface. Interestingly, combining the two enzyme systems results in changes to the substrate that suggests mechanisms of the synergistic deconstruction.

As used herein, “cellulosome complex” refers to a protein complex that utilizes a cohesin-dockerin interaction or another type of specific, non-covalent protein-protein binding even to assemble a macromolecular, multicomponent enzyme system with the ability to degrade cellulose. Cellulosome complexes may be isolated from various bacteria and fungi using the methods described in the Examples. A high molecular weight (HMW) cellulosome complex is one that is approximately 1 MDa in size. In some embodiments, a HMW cellulosome complex may be at least 0.8, 0.9, 1.0, 1.1, 1.2, 1.3, 1.4 or 1.5 MDa in size. Suitable HMW cellulosome complexes may also be greater than 1.0, 1.5 or 2.0 MDa in size.

Suitable cellulosome complexes may be from bacteria of the genera Clostridium, such C. thermocellum, C. cellulovorans, C. cellulolyticum, C. acetobutylicum, C. josui, or C. papyrosolvens, or from bacteria of the genus Acetivibrio (e.g., A. cellulolyticus), Bacteroides (e.g., B. cellulosolvens), or Ruminoccus (e.g., R. albus or R. flavefaciens). Suitable cellulosome complexes may also be from fungi of the genera Neocalimastix (e.g., N. frontalis, N. hurleyensis, N. joyonii, N. patriciarum, or N. variabilis), Piromyces (e.g., P. citronii, P. communis, P. dumbonicus, P. mae, P. minutes, P. polycephalus, P. rhizinflatus, or P. spiralis), or Orpinomyces (e.g., O. bovis or O. intercalaris).

Fungal enzymes suitable for use in the methods and combinations disclosed herein include processive and non-processive cellulases (e.g., from GH Families 5, 6, 7, 12, 45, 74, or 9), beta-glucosidases, hemicellulases, oxidoreductases (lytic polysaccharide mono-oxygenases), and other activities. β-glucosidases are a family of exocellulase enzymes that catalyze the cleavage of β(1-4) linkages in substrates such as cellobiose, resulting in the release of glucose. In some embodiments, bacterial enzymes may also be included. Endoglucanases such as the E1 endoglucanase from A. cellulolyticus may also be suitable for use in the cocktails and methods herein.

Suitable fungal enzymes may be derived from fungi of the genera Trichoderma (e.g., T. reesei, T. viride, T. koningii, or T. harzianum), Penicillium (e.g., P. funiculosum), Humicola (e.g., H. insolens), Chrysosporium (e.g., C. lucknowense), Gliocladium, Aspergillus (e.g., A. niger, A. nidulans, A. awamori, or A. aculeatus), Fusarium, Neurospora, Hypocrea (e.g., H. jecorina), and Emericella. In some embodiments, the fungal enzyme may be from H. jecorina, such as the Family 7 cellobiohydrolase Cel7A. In some embodiments, the fungal enzyme may be a commercial enzyme preparation containing one or more enzymes, such as CTec2.

The components of the fungal enzyme portion of cocktails may be varied depending on the nature of the substrate being degraded and the pretreatment protocol applied to the substrate. Exemplary fungal enzymes may comprise, by weight, 30-95% of one or more processive and non-processive cellulases such as Cel7A, 5-25% of a β-glucosidase, 5-40% of an endoglucanase such as E1, and 1-20% of additional enzymes such as xylanases (e.g., the XynA from A. cellulolyticus) or β-xylosidases. Accessory enzymes may also be included at relatively small percentages of the enzyme cocktail. A cellulase such as Cel7A may be comprise at least about 30%, 35%, 40%, 45%, 50%, 55%, 60%, 65%, 70%, 75%, 80%, 85%, 90%, or 95% of the enzyme cocktail. A β-glucosidase may comprise at least about 5%, 10%, 15%, 20%, or 25% of the enzyme cocktail. An endoglucanase such as E1 may comprise at least about 5%, 10%, 15%, 20%, 25%, 30%, 35% or 40% of the enzyme cocktail. A xylanase or β-xylosidase may comprise at least about 1%, 2%, 3%, 4%, 5%, 6%, 7%, 8%, 9%, 10%, 11%, 12%, 13%, 14%, 15%, 16%, 17%, 18%, 19% or 20% of the enzyme cocktail. Additional accessory enzymes may comprise at least about 1%, 2%, 3%, 4%, 5%, 6%, 7%, 8%, 9% or 10% of the enzyme cocktail.

The relative amounts of fungal enzymes and cellulosomes in an enzyme cocktail may also be varied depending on the nature of the substrate being degraded and the pretreatment protocol applied to the substrate. For example, synergistic activity is seen when the cellulosomal portion of the cocktail makes up about 25% of the total cocktail by weight. The cellulosomal component may be present in weight percentages of at least about 10%, 15%, 20%, 25%, 30%, 35%, 40%, 45%, 50%, 55%, 60%, 65%, 70%, 75%, 80%, 85%, or 90% of the total cocktail. In certain embodiments, the free enzyme component may be present in weight percentages of at least about 10%, 15%, 20%, 25%, 30%, 35%, 40%, 45%, 50%, 55%, 60%, 65%, 70%, 75%, 80%, 85%, or 90% of the total cocktail. Exemplary cocktails comprise ratios of 25:75, 50:50, and 75:25 of cellulosome and free enzyme, respectively, by weight.

The enzyme and cellulosome cocktails exhibit surprisingly improved cellulase activities when compared to the individual enzyme or cellulosome activities or the additive effect of each enzyme or cellulosome. The term “improved activity” refers to an increased rate of conversion of a cellulosic substrate or a specific component thereof. Relative activities can be determined using conventional assays, including those discussed in the Examples below. Additional assays suitable for determining cellulase activity include hydrolysis assays on industrially relevant cellulose-containing substrates such as pretreated corn stover. Hydrolysis assays on crystalline cellulose or amorphous cellulose or on small molecule fluorescent reporters may also be used to determine cellulase activity. In certain embodiments, cellulase activity is expressed as the amount of time or enzyme concentration needed to reach a certain percentage (e.g., 80%) of cellulose conversion to sugars.

Enzymes described herein may be used as purified recombinant enzyme or as culture broths from cells that naturally produce the enzyme or that have been engineered to produce the enzyme. Cellulosomes are traditionally purified from culture broths, but can also be made using recombinant DNA technology or by using raw culture broths. In certain embodiments, enzyme cocktails may achieve cellulose conversions to sugars (as a percentage of the total cellulose in the original substrate) ranging from 50% to 100%, 70% to 100%, or 90% to 100%. In some embodiments, the cellulose conversion exhibited by the enzyme cocktail may be at least about 50%, 55%, 60%, 65%, 70%, 75%, 80%, 85%, 90%, 95%, or 99%.

Methods for degrading cellulose and materials containing cellulose using the enzyme cocktails are also provided herein. For example, the enzyme cocktails may be used in compositions to help degrade (e.g., by liquefaction) a variety of cellulose products (e.g., paper, cotton, etc.) in landfills. The enzyme cocktails may also be used to enhance the cleaning ability of detergents, function as a softening agent or improve the feel of cotton fabrics (e.g., stone washing or biopolishing) or in feed compositions.

Cellulose containing materials may also be degraded to sugars using the enzyme cocktails. Ethanol may be subsequently produced from the fermentation of sugars derived from the cellulosic materials. Exemplary cellulose-containing materials include bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, corn fiber, grasses, wheat, wheat straw, barley, barley straw, hay, rice straw, switchgrass, waste paper, sugar cane bagasse, sorghum, soy, components obtained from milling of grains, trees, branches, roots, leaves, wood (e.g., poplar) chips, sawdust, shrubs and bushes, vegetables, fruits, flowers and animal manure.

Biofuels such as ethanol may be produced by saccharification and fermentation of lignocellulosic biomass such as trees, herbaceous plants, municipal solid waste and agricultural and forestry residues. Typically, saccharification is carried out by contacting the lignocellulosic biomass with an enzyme cocktail that includes one or more of the enzymes or cellulosomes described herein. Such enzyme cocktails may also contain one or more endoglucanases (such as the Family 5 endoglucanase E1 from Acidothermus cellulolyticus) or one or more β-glucosidases (e.g., a β-glucosidase from A. niger) to optimize hydrolysis of the lignocelluloses. Additional suitable endoglucanases include EGI, EGII, EGIII, EGIV, EGV or Cel7B (e.g., Cel7B from T. reesei). Enzyme cocktails may also include accessory enzymes such as hemicellulases, pectinases, oxidative enzymes, and the like.

Enzymes with the ability to degrade carbohydrate-containing materials, such as cellulases with endoglucanase activity, exoglucanase activity, or β-glucosidase activity, or hemicellulases with endoxylanase activity, exoxylanase activity, or β-xylosidase activity may be included in enzyme cocktails. Examples include enzymes that possess cellobiohydrolase, α-glucosidase, xylanase, β-xylosidase, α-galactosidase, β-galactosidase, α-amylase, glucoamylases, arabinofuranosidase, mannanase, β-mannosidase, pectinase, acetyl xylan esterase, acetyl mannan esterase, ferulic acid esterase, coumaric acid esterase, pectin methyl esterase, laminarinase, xyloglucanase, galactanase, glucoamylase, pectate lyase, chitinase, exo-β-D-glucosaminidase, cellobiose dehydrogenase, ligninase, amylase, glucuronidase, ferulic acid esterase, pectin methyl esterase, arabinase, lipase, glucosidase or glucomannanase activities.

A lignocellulosic biomass or other cellulosic feedstock may be subjected to pretreatment at an elevated temperature in the presence of a dilute acid, concentrated acid or dilute alkali solution for a time sufficient to at least partially hydrolyze the hemicellulose components before adding the enzyme cocktail. Additional suitable pretreatment regimens include ammonia fiber expansion (AFEX), treatment with hot water or steam, or lime pretreatment.

Lignocellulosic biomass and other cellulose containing materials are contacted with enzyme cocktails at a concentration and a temperature for a time sufficient to achieve the desired amount of cellulose degradation. The enzyme cocktails disclosed herein may be used at any temperature, but are well suited for digestions around 50 or 60° C. For example, the enzymes or cocktails may be used at temperatures ranging from about 40° C. to about 60° C., from about 50° C. to about 55° C., from about 50° C. to about 60° C., or from about 45° C. to about 55° C. Exemplary temperatures include 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70° C.

Suitable times for cellulose degradation range from a few hours to several days, and may be selected to achieve a desired amount of degradation. Exemplary digestion times include 1, 2, 3, 4, 5, 6, 7, 8, 9, 10, 11, or 12 hours; and 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5 or 15 days. In some embodiments, digestion times may be one or more weeks.

Separate saccharification and fermentation is a process whereby cellulose present in biomass is converted to glucose that is subsequently converted to ethanol by yeast or bacteria strains. Simultaneous saccharification and fermentation is a process whereby cellulose present in biomass is converted to glucose and, at the same time and in the same reactor, converted into ethanol by yeast or bacteria strains. Enzyme cocktails may be added to the biomass prior to or at the same time as the addition of a fermentative organism.

The resulting products after cellulose degradation may also be converted to products other than ethanol. Examples include conversion to higher alcohols, hydrocarbons, or other advanced fuels via biological or chemical pathways, or combination thereof.

The free enzyme cocktail exemplified here is primarily comprised of components with a single catalytic unit and single CBM per protein. This represents the simplest free enzyme “unit”, which can in different organisms range from single catalytic units and single CBMs up to several catalytic units and CBMs per protein. Conversely, the cellulosomal system exemplified here contains from 9 to 30 catalytic units and multiple CBMs per individual complex linked via cohesin-dockerin interactions. These two examples of free and cellulosomal enzyme cocktails are among the primary types of enzyme cocktail candidates for industrial application in the growing biofuels industry. Combining these two systems demonstrates how these two systems are complementary.

Typically, cellulosomes are more efficient at digesting crystalline cellulose, while the cellulosomes are not as effective as the free enzymes at degrading pretreated biomass, yet together the two systems are more efficient at cellulose degradation. TEM images reveal a novel mechanism by which cellulosomes are able to increase the accessible surface area for degradation in substrates such as Avicel by a factor of 2 over free enzymes by separating individual cellulose microfibrils from larger particles. The free enzymes, in contrast, primarily act via an ablative mechanism. These results suggest that free and complexed enzymes act via different mechanisms, and over different critical length scales. In combination, these two mechanisms can act synergistically to deconstruct cellulose.

One possible factor that contributes to the lower activity of cellulosomes on the pretreated substrates is that the large cellulosomes are unable to separate individual cellulose microfibrils from biomass particles. This may be due to the presence of other plant cell wall components including lignin and residual hemicellulose. The cellulosomes, because of their size relative to the free enzymes, may not be able to gain access to as many of the microfibril-microfibril contact surfaces in pretreated biomass, thus limiting their ability to separate individual cellulose microfibrils for localized attack.

Another possibility that may contribute to the differences between free and complexed enzymes on pretreated biomass is the trapping of enzymes by non-specific binding to residual lignin. This trapping process would progressively remove enzymes from the population able to digest cellulose. Such an inactivation mechanism would almost certainly, affect the fungal enzymes as well as the cellulosomes, but the much higher molecular weight of the cellulosome implies that for equal loadings on a mass basis, the cellulosomes would be present in much lower molar concentrations. Each nonproductive binding event would therefore be expected to have a more substantial negative effect on activity in the case of the cellulosomal system.

Individually, the free and complexed enzymes digest Avicel particles from a single end. Many fungal cellulase cocktails rely on a reducing-end specific, Family 7 cellobiohydrolase for significant hydrolytic potential, and it is therefore likely that the reducing end of the cellulose bundles is where free cellulases attack. Similarly, cellulosomes are known to produce processive Family 48 cellobiohydrolases, such as C. thermocellum CelS, in abundance. Family 48 cellobiohydrolases also act from the reducing end of cellulose, and have been shown to be key enzymes in cellulosomal activity. Yet when acting together, free cellulases and cellulosomes function throughout the cellulose particle by increasing the available reactive surface area, which allows for the penetration of enzyme accessibility and degradation of exposed ends.

Free enzymes with a single catalytic unit per protein and huge cellulosomal enzyme complexes function via different physical mechanisms to deconstruct recalcitrant polysaccharides, despite employing similar component enzymes and CBMs. The results disclosed herein suggest that cellulosomes work by separating individual microfibrils from large cellulose particles, which allows for localized enzymatic attack. Conversely, the free enzymes examined here display a longer critical length scale for ablative action down single microfibrils that are available for CBM binding, and hence sharpen both the cellulose particles and individual cellulose microfibrils simultaneously. Smaller enzyme complexes with multiple catalytic units and possibly multiple CBMs per protein (but smaller than the cellulosome) may employ strategies with characteristics of both mechanisms.

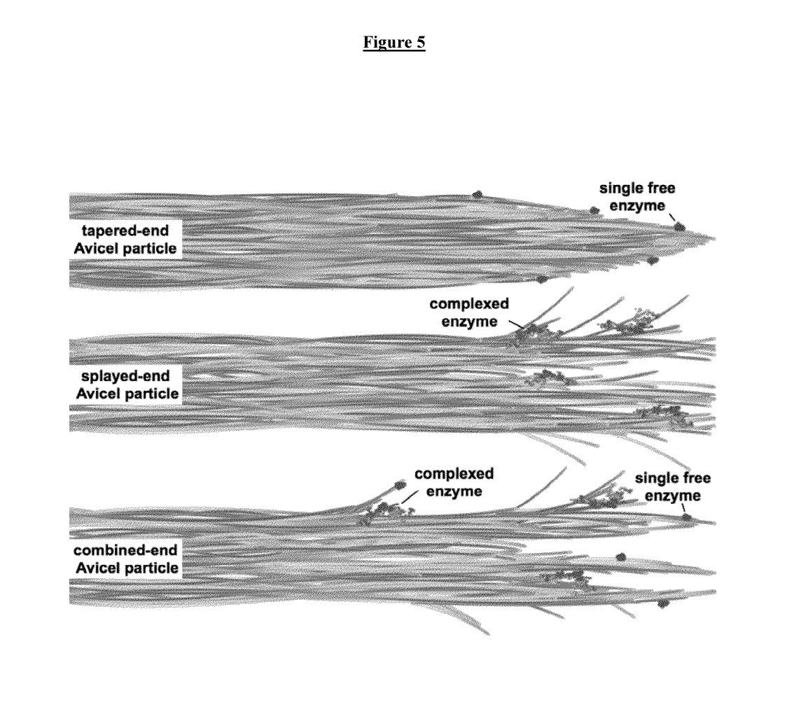

Without wishing to be bound by any particular theory, it is believed that free enzymes with one catalytic unit and a single CBM may be restricted to digesting only the surface of the crystalline cellulose microfibril bundles. The higher activity and processivity of the reducing-end-active enzymes in the complex would lead to the overall tapered morphology. Conversely, complexed enzymes with multiple CBMs may be able to exploit the occasional loosening within bundles by binding several microfibrils at once, maintaining the space between them, and thereby increasing the total substrate surface area available to enzymatic digestion. Accessibility to free microfibril ends that could be splayed would be limited in whole biomass by the presence of lignin and hemicellulose, which could explain why the performance of cellulosomes on intact biomass is compromised. A schematic of these principles is presented in FIG. 5.

### EXAMPLES

### Example 1

The following materials and methods were used in subsequent Examples detailed below.

Isolation of the Secretome and the Cellulosome Enriched Sample from C. thermocellum

C. thermocellum was grown on Avicel PH101. The secretome was separated from the cellular debris by centrifuging the cells at 12,000×g for 30 minutes at 4° C. The cellulosome-enriched sample was isolated by ammonia sulfate precipitation. After the ammonium sulfate dissolved completely, a precipitate slowly formed and was collected by centrifugation (about 8° C., 7000 RPM, 15 minutes). The supernatant fluids were discarded and the pellet fraction was dissolved in PBS (about 300 mL). The clarified supernatant, enriched with cellulosomal and non-cellulosomal components, was filtered via a 0.2 micron filter. The filtrate was then applied to an ultrafiltration device with a nominal molecular-weight cut-off of 300K (Millipore) at 4° C. After reduction of the solution to about 200 mL, the concentrate was analyzed by SDS-PAGE. The cellulosome-enriched secretome was then dialyzed against Tris buffered saline (0.1 M Tris-HCl, 0.15 M NaCl, pH 7.2) overnight at 4° C. (3 L volume×4 buffer changes). Protein concentration for the cellulosome-enriched sample was estimated spectrophotometrically by the Bradford method (BSA as standard protein). The sample was divided into 15 mL tubes and stored at −20° C.

### Fractionation of HMW Cellulosomes

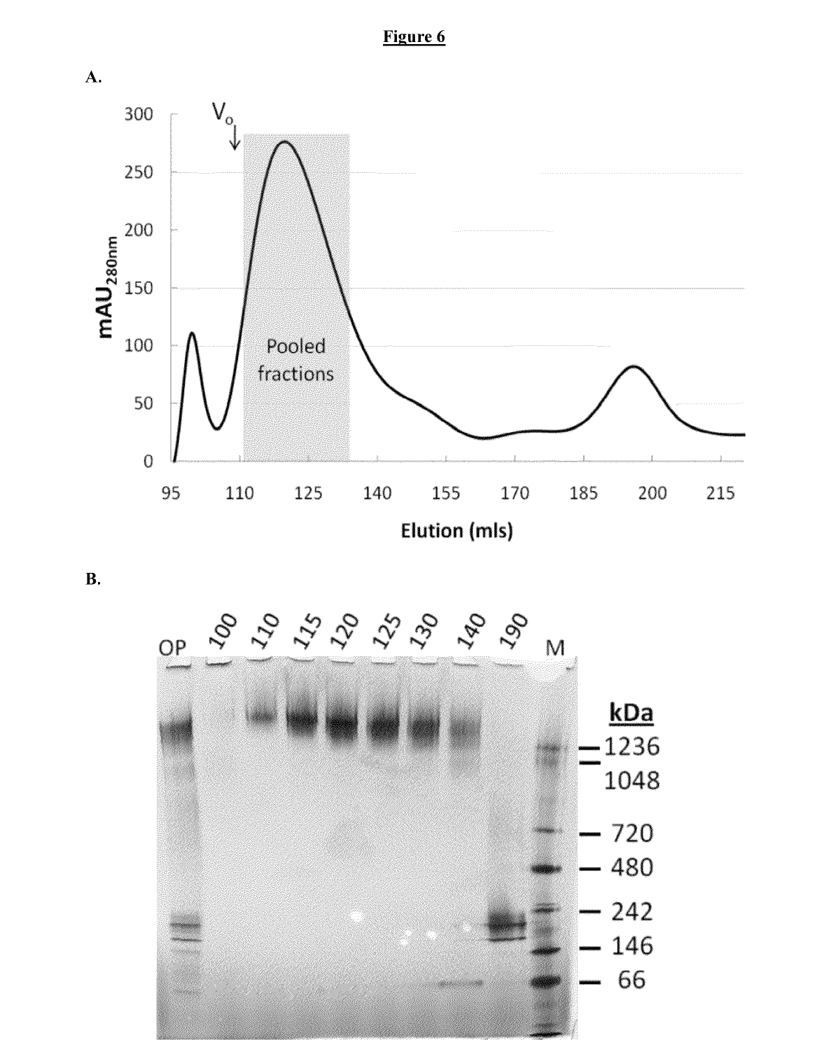

5 mL of the cellulosome-enriched sample was loaded on a Sephacryl S-400 26/60 SEC column (GE) to purify the high molecular weight (HMW) cellulosomes based on the method of Lamed et al., Enzyme Microb Technol 7(1):37-41 (1985). Separation was run at 1.5 mL per minute and 0.5 mL fractions were collected. Elution was monitored by the absorption at 280 nm. Fractions were collected and analyzed using denaturing and native poly-acrylamide gel electrophoresis (PAGE) to identify the cellulosome-containing fractions (FIG. 6).

### Poly-Acrylamide Gel Electrophoresis

Ten micrograms of each sample was loaded on Native-PAGE Novex Bis-tris 3-12% gels (Invitrogen) that utilize G-250 compound to eliminate the protein charge effect on electrophoretic migration. Gels were stained with Coomassie Blue protein stain (Invitrogen) and imaged on an HP image scanner (Hewlett Packard).

### Hemicellulase Enzyme Purification

Hemicellulase enzyme genes from A. niger, AbfB, XynA, and XlnD, were transformed into A. nidulans. The gene from P. funiculosum FaeA was transformed into A. nidulans. The gene from T. reesei FaeA was transformed into A. nidulans. All of the hemicellulase genes were expressed in the aforementioned expression hosts and purified chromatographically.

### Fungal Cellulases

CTec2 was obtained from Novozymes in a solution containing about 210 mg/mL of protein as measured using the BCA protein determination kit (Pierce) after desalting. The concentrated enzyme mixture was applied to an AKTA FPLC (GE) using a HiPrep 26/10 Sephadex (GE) desalting column to remove stabilizers and other additives that interfere with HPLC analysis of digested biomass and cellulose.

### Cellulose Substrates

Whatman #1 filter paper was cut into 14-mg pieces and suspended in double-distilled H₂0 under vacuum overnight at 40° C. then washed three times with buffer containing 30 mM Na-Acetate, pH 5.0, 0.001% Na-azide prior to enzymatic assays.

Phosphoric acid swollen cellulose (PASC) was prepared from Sigmacell cellulose type 50 (Sigma-Aldrich). Five grams of Sigmacell was first moistened with deionized water, then 150 mL of 85% phosphoric acid was added slowly over a period of 1 hour with gentle stirring while the slurry was maintained at 40° C. After the addition of 100 mL of cold acetone, the slurry was centrifuged for 10 minutes at 5000×g. The pellet was washed three times by resuspension and centrifugation in deionized water.

Avicel PH101 was suspended in double-distilled water overnight and washed three times with double distilled water by centrifugation at 500×g. The pellets were resuspended to a concentration of 20 mg/mL (w/w) in 30 mM Na-acetate buffer, pH 5.0, containing 0.001% (w/v) sodium azide.

Poplar and switchgrass biomass were pretreated in a continuous reactor. The switchgrass was pretreated at 1900° C. with a sulfuric acid loading of 50 mg/g dry solids, at an estimated residence time of 1 minute. The solids loading in the pretreatment reactor was 25% (w/w). Poplar pretreatment conditions were 1950° C., 30 mg acid/g dry biomass, 1 minute residence time and 25% (w/w) total solids.

### Activity Assays

Cellulosomal enzyme activity was determined at 60° C. and pH 5.0 in 20 mM Na-acetate buffer containing 10 mM CaCl₂, 100 mM NaCl, 2 mM EDTA and 10 mM cysteine. Fungal cellulase (CTec2) activity was measured at 50° C. in 20 mM Na-acetate, pH 5.0. All digestions were carried out in sealed 2 mL HPLC vials with continuous mixing by inversion at 10/minute. Unless otherwise noted, substrates were loaded at 10 mg cellulose per mL of digestion mixture, with enzymes in turn loaded at 10 mg of cellulase protein per g of glucan in 1.4 mL reaction volumes. Representative (with respect to both solid and liquid phases of the digestion slurry) 0.1 mL samples were withdrawn from well-mixed digestion mixtures at selected time-points during the digestions and diluted 10-fold with deionized water into 2.0 mL HPLC vials that were then crimp-sealed and immersed in a boiling-water bath for 10 minutes to inactivate the enzymes and terminate the reaction. The diluted and terminated digestion aliquots were then filtered through 0.2 μm nominal-pore-size nylon syringe-filters (Pall/Gelman Acrodisc-13) to remove residual substrate and most of the denatured enzyme. Released soluble sugars in the diluted samples were then determined by HPLC analysis on an Aminex HPX-87H column (Bio-Rad Laboratories, Inc., Hercules, Calif., USA) operated at 65° C. with 0.01 N H₂SO4 as mobile phase at 0.6 mL/minute in an Agilent 1100 HPLC system with refractive-index detection. The resulting glucose and cellobiose concentrations calculated (in mg/mL) for each digestion mixture were then converted to anhydro-glucose and anhydro-cellobiose concentrations, respectively, by subtracting out the proportional weight added to each molecule by the water of hydrolysis. The sum of the concentrations of anhydro-glucose and anhydro-cellobiose, which sum is equivalent to the weight-concentration of the glucan chain that was hydrolyzed to produce the soluble sugars, was then divided by the initial weight-concentration of cellulose in the digestion mixture and multiplied by 100% to yield activity results as percent conversion of cellulose.

### TEM Sample Preparation and Imaging

Digested Avicel PH101 samples were drop cast directly on 0.35% Formvar-coated slot grids and negatively stained with 2% aqueous uranyl acetate.

For immuno-EM, grids were placed on 10 μL drops of 2.5% non-fat dry milk in 1X PBS-0.1% Tween (PBST) for 30 minutes, then directly placed on about 10 μL drops, on parafilm, of primary antibodies diluted 1:50 in 1% milk PBST and incubated overnight at 4° C. Following 3×1 minute rinses, grids were placed on 10 μL drops of secondary antibody-15 nm gold conjugate (British BioCell) diluted 1:100 in PBST. Grids were then rinsed 3×1 minutes with PBST followed by H₂O.

Pretreated and digested switchgrass samples were high-pressure frozen in 0.2 mm brass planchettes in a Leica EM PACT2 (Leica Microsystems GmbH, Wetzlar, Germany). Planchettes were placed in cryovials and freeze substitution took place in a Leica AFS2 automatic freeze substitution unit in 2.5% glutaraldehyde (w/v), 0.1% uranyl acetate (w/v) acetone for 4 days at −90° C., increasing the temperature to −30° C. over 24 hours, then increasing the temperature to 3° C. over 24 hours. The fixation solution was replaced with 100% acetone and the temperature was then brought to 18° C. over 1 hour. Samples were washed 3 times in 100% acetone for 1 hour, removed from their hats, and placed in BEEM capsules (BEEM Inc., Bronx, N.Y.). LR White resin (EMS, Hatfield, Pa.) was added to the sample capsules in the following concentrations (v/v) acetone: 25%, 50%, 75%, and 100%×3 for 1 day each. Samples were then incubated in 100% LR White for 6 hours and polymerized for 24 hours at 60° C. in a nitrogen-purged vacuum oven.

Sectioning was performed with a Diatome diamond knife (EMS, Hatfield, Pa.) on a Leica EM UTC ultramicrotome (Leica Microsystems GmbH, Wetzlar, Germany). All embedded samples were sectioned to a thickness of approximately 60 nm and collected on 0.35% Formvar-coated palladium/copper slot grids (SPI Supplies, West Chester, Pa.). Grids were post-stained for 4 minutes with 2% aqueous uranyl acetate and 2 minutes in 1% KMnO₄ to enhance lignin staining. Images were taken with a 4 mega-pixel Gatan UltraScan 1000 camera (Gatan, Pleasanton, Calif.) on a FEI Tecnai G2 20 Twin 200 kV LaB6 TEM (FEI, Hilsboro, Oreg.).

Fiji (ImageJ) was used to perform image analysis on the TEM micrographs to calculate the 2D perimeter of the digested Avicel particles as an estimation of the actual 3D exposed surface area. Briefly, micrographs were opened in Fiji and a region of interest measured 1 μm from the tapered or splayed end was thresholded to delineate the particle from the background carbon film. The thresholded image was converted to binary. Binary image process operators were used to ensure that a single Avicel particle was represented. These operators included one iteration for one count of the Close operation and one iteration of the Fill Holes tool. The Analyze Particles tool was then used to report the perimeter of the binary object within the defined region of interest.

### Example 2

### Cellulosome Activity Enrichment by Purification

The cellulosome preparation was produced from the culture-filtrate of C. thermocellum by successive affinity-selection (binding to microcrystalline cellulose), elution from the cellulose with 1% triethylamine, concentration over a 300 kDa nominal-pore-size ultrafiltration membrane to produce a “cellulosome-enriched secretome” (Ces), which was then further fractionated by size-exclusion chromatography, as described below. The cellulosome enzyme complex was enriched from the secretome that had been ammonium-sulfate precipitated and selected for protein material above 300 kDa.

Free cellulases, non-cellulolytic proteins and aggregated proteins are present along with the cellulosomes in the extracellular growth medium. Size exclusion chromatography (SEC) was used to separate the high molecular weight (HMW, >1MDa) cellulosomes from the non-cellulosomal (smaller) and aggregated (much larger) proteins. FIG. 6A shows size exclusion chromatography (SEC) purification of High Molecular Weight (HMW) cellulosomes. Concentrated cellulosome-enriched broth was applied to a HiPrep Sephacryl HR S400 26/60 SEC column (GE) to separate HMW cellulosomes (>1MDa) (110-135 mL elution volume) from protein aggregates (95-110 mL) and free enzymes (>140 mL). Eluted fractions were analyzed by native PAGE on a Novex 3-12% Bis-Tris Gel (Invitrogen), as shown in FIG. 6B. Fractions in the elution volumes between 112-135 mL (grey box) were pooled and concentrated to about 1 mg/mL. The fractions that eluted between 112 and 135 mL contained a discrete HMW cellulosome band and were pooled and concentrated to 1 mg/mL for enzymatic characterization.

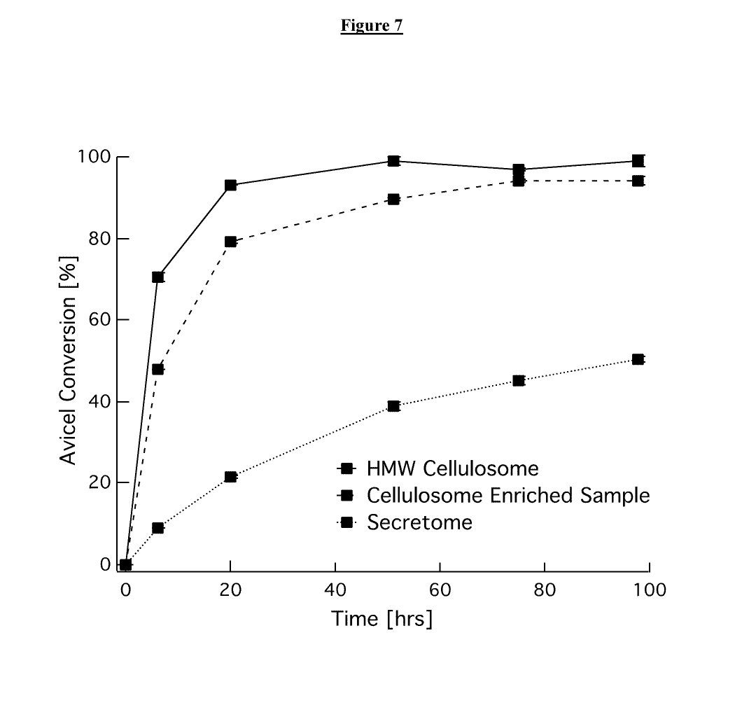

C. thermocellum secretome, cellulosome enriched cellulosome mixture and the HMW cellulosomes were compared for their enzymatic activity on Avicel. The enzymes mixtures were loaded equally at 10 mg/g (10 mg of protein was loaded per g of Avicel in a 1% solids loading). FIG. 7 shows the enzymatic digestion of Avicel PH-101 by SEC purified cellulosomes (−), cellulosome-enriched secretome (CES) ( - - - ), and C. thermocellum secretome ( . . . ). The results shown in FIG. 7 illustrate that purification of the HMW cellulosome increases the specific activity. This enrichment improved the activity by 14% compared to the cellulosome enriched secretome and 4-fold with respect to the secretome at 20 hours into the digestion.

### Example 3

### Optimization of Cellulosome Enzymatic Activity Conditions

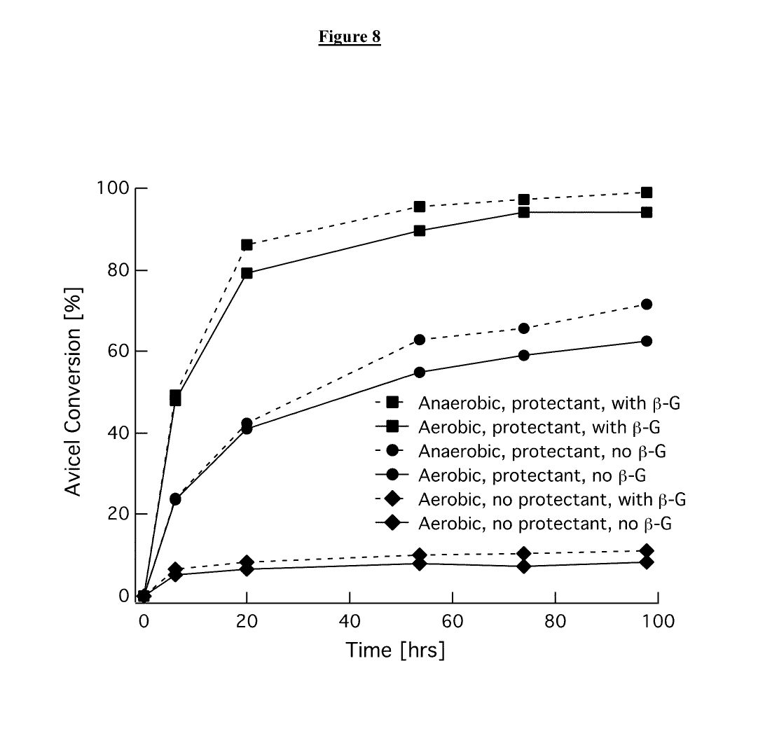

FIG. 8 shows the overall results for the optimization of cellulosome enzymatic activity on Avicel as a function of aerobic or anaerobic conditions, O-glucosidase presence, and the presence of chemical protectants. Some enzymes of the C. thermocellum cellulosome are oxygen sensitive, so cysteine was used as a reducing agent to remove oxygen from the enzymatic assays. FIG. 8 shows the saccharification of Avicel by the HMW cellulosomes assayed with or without cysteine protectant. In the absence of cysteine, we observe low Avicel conversion, which is consistent with the observation that certain cellulosomal enzymes are subject to oxygen-induced inactivation. The optimized conditions were used in the remainder of experiments in this study for activity assay of cellulosomes.

Additionally, calcium has been shown to stabilize cohesin-dockerin interactions and other cellulosomal domains, and it is necessary for catalysis in some cellulosomal enzymes. Ten mM CaCl₂ was maintained in all assays, and EDTA was added (with Ca²⁺ kept in molar excess over the EDTA) to scavenge trace amounts of other transition metal ions that may promote oxidation. Lastly, because many cellulases, including important members of the cellulosomal array are inhibited by cellobiose, O-glucosidase that had been chromatographically purified from a commercial Aspergillus niger preparation (Novozym 188, Novozymes USA) was added to mitigate product inhibition.

The addition of both sulfhydryl-protectants and β-glucosidase results in a marked increase in activity in comparison with the activity observed in the absence of either of these two adjuvants In addition to the effects of adding protectants and a O-glucosidase, slightly higher sustained activities are attained when the digestions are conducted in anaerobic conditions (FIG. 8). The differences in activity observed between the anaerobic and aerobic assays, however, are small compared to the differences between the reactions with and without protectants and β-glucosidase.

### Example 4

### Role of Hemicellulase Enzymes in Biomass Conversion by Cellulosomes

Cellulosomes are known to contain hemicellulase enzymes. To explore the hypothesis that hemicellulase enzymes associated with the cellulosome are insufficient to enable effective degradation of complex cell wall carbohydrates, purified hemicellulases were added to the reaction mixture along with the cellulosome in assays against pretreated switchgrass. The following hemicellulase enzymes were used: acetylxylan esterase (Axe), arabiofuranosidase (AbfB), ferulic acid esterase (Fae), β-1,4 xylanase (XynA), and xylobiase (XylD). The total amount of protein loaded was 10 mg of protein per g of cellulose. The “cellulosome only” reaction contained 10 mg/g of purified cellulosome. The combined reaction contained 5 mg/g of the HMW cellulosome and five hemicellulases loaded at 1 mg/g of each hemicellulase (Axe, AbfB, XynA, XylD, and Fae; - - - ) for a total of 10 mg of protein. The reactions were loaded with 1% solids and incubated at 60° C. in buffer containing 25 mM Na—Ac, pH 5.0, 100 mM NaCl, 10 mM Cysteine, 10 mM CaCl₂, 2 mM EDTA and 2 mg/g β-glucosidase.

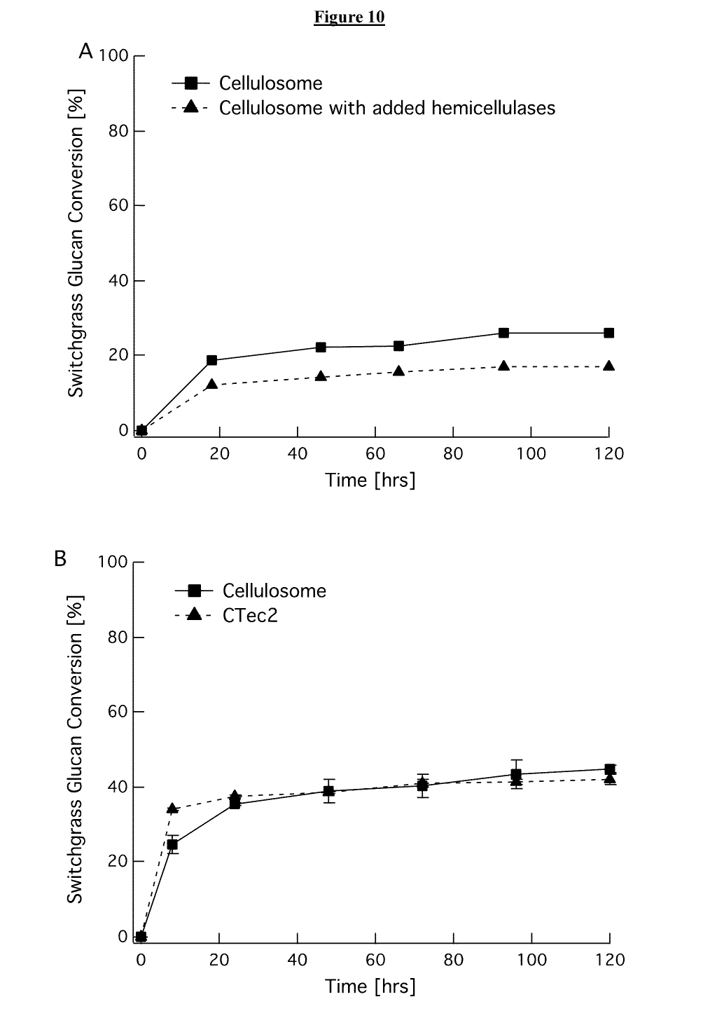

We found that supplementing the cellulosome loading with hemicellulases actually reduced the overall conversion (FIG. 10A). This result confirms that lowering the cellulosome loading reduces the conversion, and indicates that the poor conversion by the cellulosome is not due a deficiency of hemicellulases.

### Example 5

### The Ability of Free and Complexed Enzymes to Digest Untreated Switchgrass

To test the digestion of biomass that has not been treated with chemicals or high temperatures, 50 mg/g of cellulosomes and CTec2 was used in enzymatic degradations to measure the glucose and cellobiose release. The reactions were loaded with 1% solids and incubated at 60° C. in buffer containing 25 mM Na—Ac, pH 5.0, 100 mM NaCl, 10 mM Cysteine, 10 mM CaCl₂, 2 mM EDTA and 2 mg/g β-glucosidase. Surprisingly the conversion was identical using the enzymatic systems (FIG. 10B). The two enzyme systems thus appear to have a similar ability to degrade the accessible cellulose.

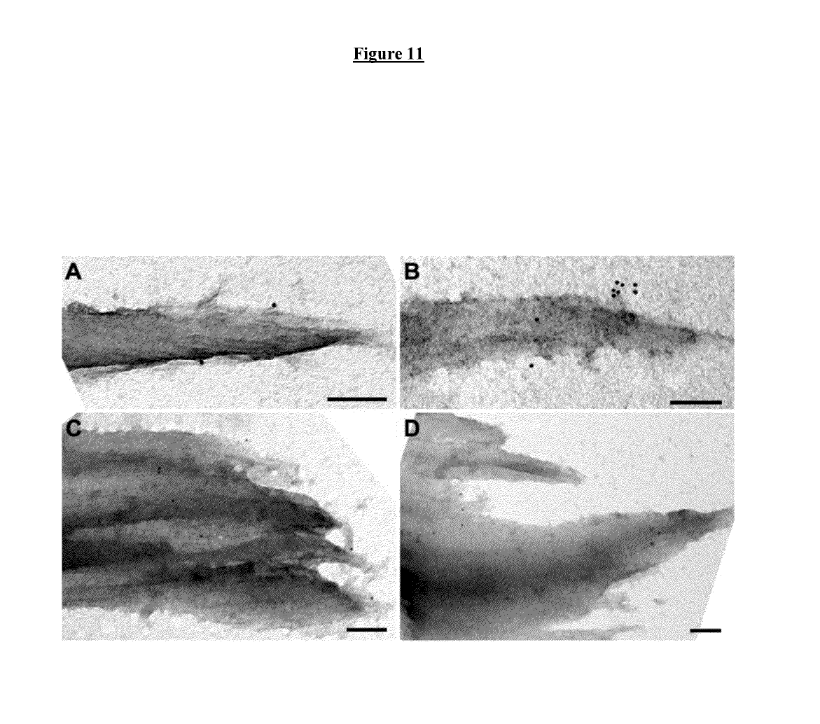

The morphological changes in the cell wall architecture of pretreated switchgrass biomass particles digested by either free fungal cellulase enzymes or high molecular weight cellulosomes was also examined. These samples were preserved by high-pressure freezing and freeze substitution to generate the highest possible structural preservation and to retain the antigenicity of enzyme epitopes for immuno-localization studies. The samples were immuno-labeled with 15 nm gold conjugated antibodies that appear as black spots on the micrographs to localize Cel7A enzymes (FIGS. 11A and B) or the cellulosome scaffoldin protein (FIGS. 11C and D) on or within the cellulose microfibril bundles. The pretreated samples imaged before any exposure to cellulosomes or fungal free enzymes were already extensively fractured and delaminated due to the milling and dilute acid pretreatments (FIGS. 11A and B). These morphological changes are characteristic of dilute acid pretreated biomass. In order to visualize the changes in the pretreated biomass particle structure during digestion by either the CTec2 enzyme cocktail or cellulosomes purified from Clostridium thermocellum, samples from the digestion reactions were taken at 0, 4, and 24 hours for ultrastructural observation.

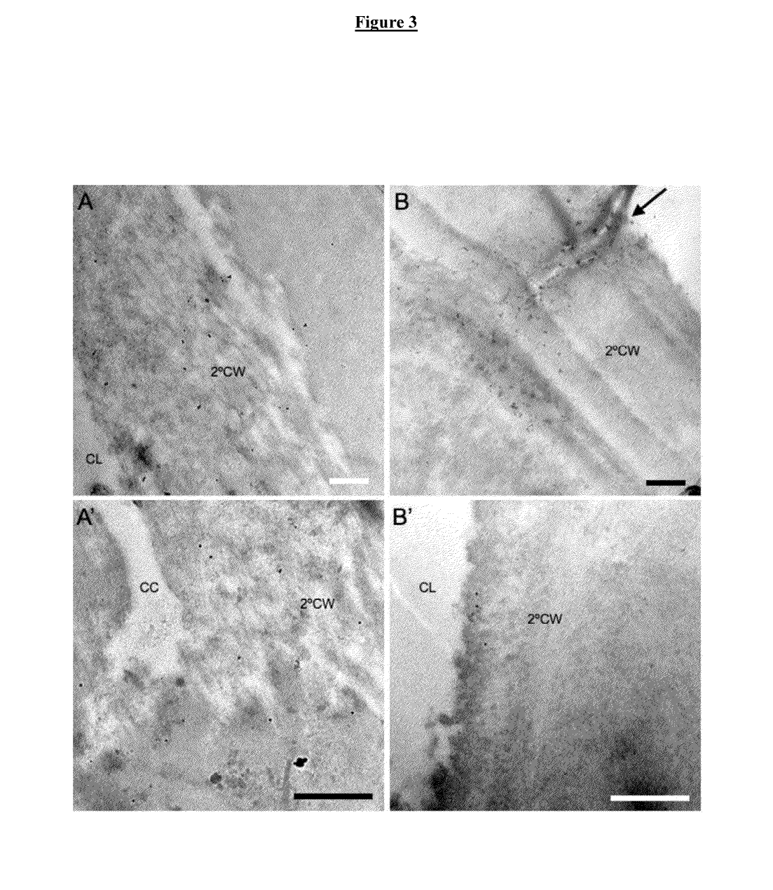

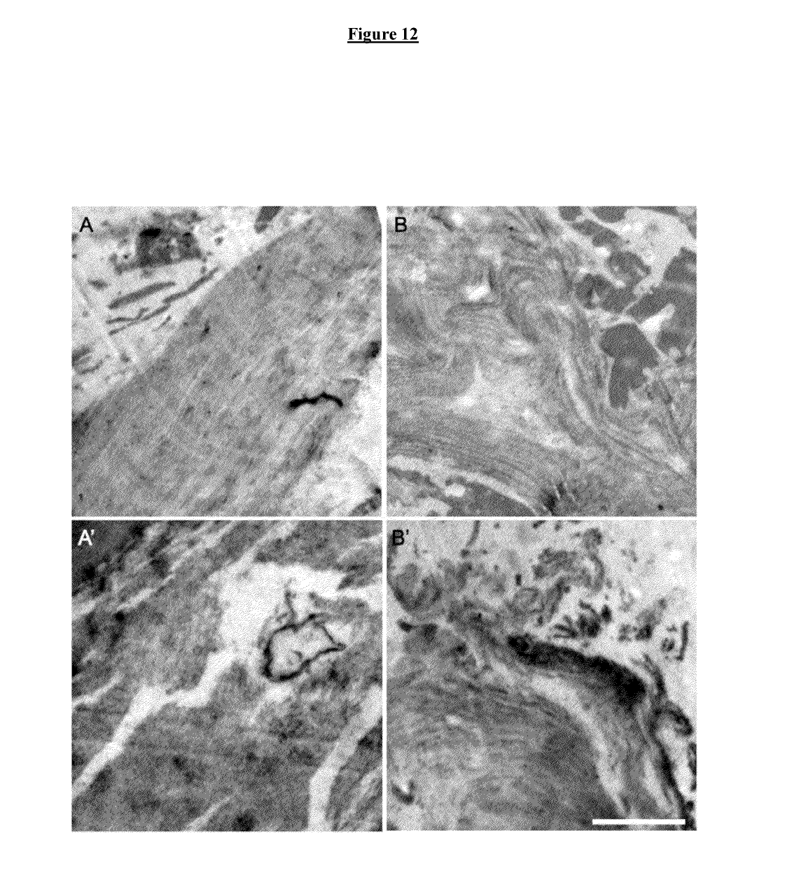

TEM micrographs at two different magnifications are presented in FIGS. 11 and 12. As shown in FIG. 12, the dilute acid pretreated biomass particles (A, B) display extensive fracturing and delamination within the cell walls from the milling and pretreatment process. The pretreated particles digested for 24 hours with CTec2 (A′) or with cellulosomes for 24 hours (B′) displayed extensive variability in cell wall morphology and patterns of deconstruction. There were not obvious differences in the morphological properties of the biomass cell walls that would explain the difference in the performance of free versus complexed enzymes. The images of these digested biomass samples suggests that there is variability within each sample and that it was not easy to determine consistent morphological properties that distinguish the CTec2 digested samples from the cellulosome digested samples. However, the immuno-localization of enzyme penetration into the pretreated biomass particles does give some insight into enzyme localization (FIG. 3).

### Example 6

### Development of Optimal Reaction Conditions for Cellulosome Activity

To compare enzymatic activity between the free and complexed enzyme systems in an unbiased manner, high molecular weight (HMW) cellulosomes were isolated from the aggregated and free proteins in the extracellular media, and then the activity of the HMW cellulosomes was optimized. For the isolation procedure, affinity purification and size exclusion chromatography (SEC) was used to separate the HMW (>1MDa) cellulosomes from the non-cellulosomal and aggregated proteins. FIG. 6A shows the separation and pooling of fractions containing the HMW cellulosomes. SEC purification of the HMW cellulosome from the original broth increased the Avicel conversion significantly compared to that of the entire secretome (FIG. 7). This purified cellulosomal fraction (HMW) was used in the Examples described below.

Three variables known to influence C. thermocellum cellulosome enzymatic activity and complex stability were examined to optimize the reaction conditions of cellulosomes: oxygen sensitivity, stabilization by the presence of calcium, and product inhibition by cellobiose. FIG. 8 shows the overall results for the optimization of cellulosome enzymatic activity as a function of these three factors. Cellulosome digestion conditions contained L-cysteine as reducing agent, CaCl₂ and β-glucosidase were found to be most optimal.

### Example 7

### Degradation of Crystalline Cellulose

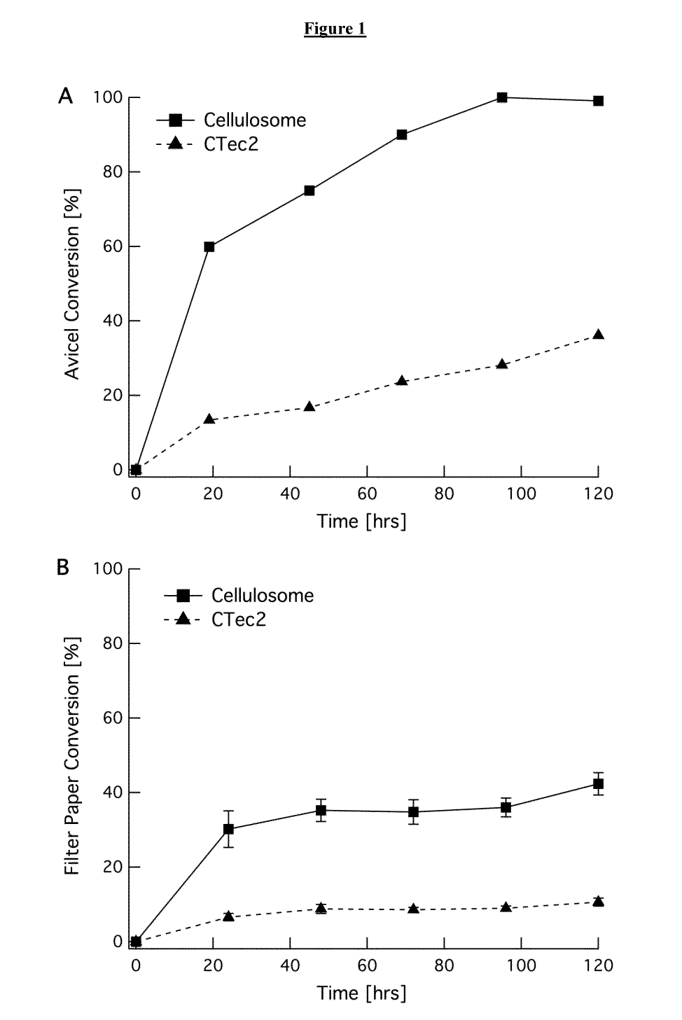

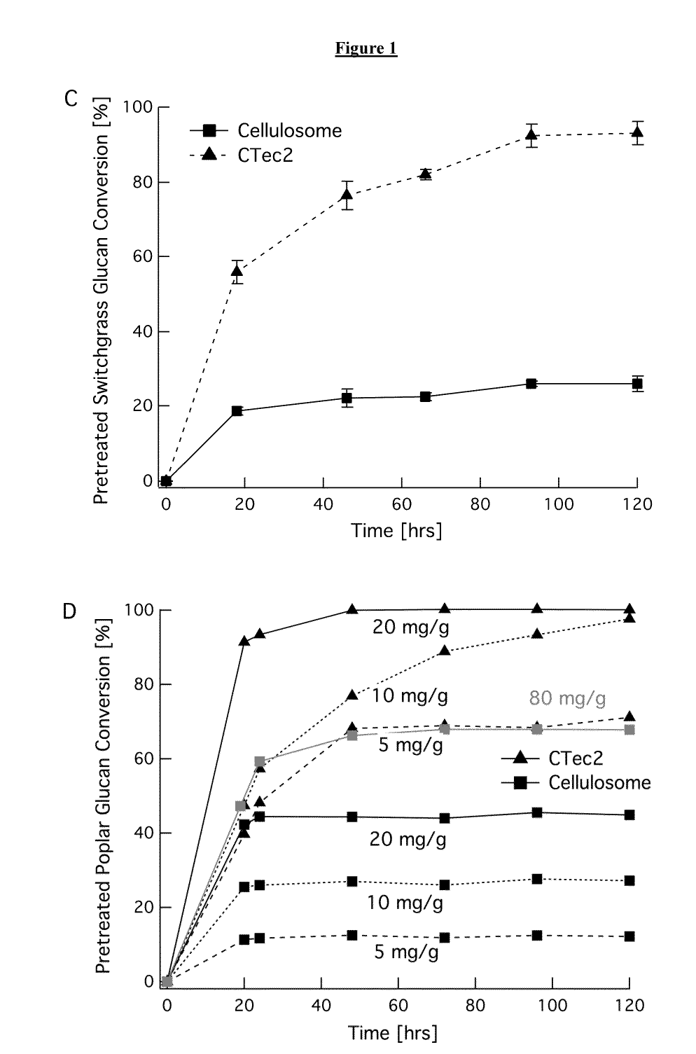

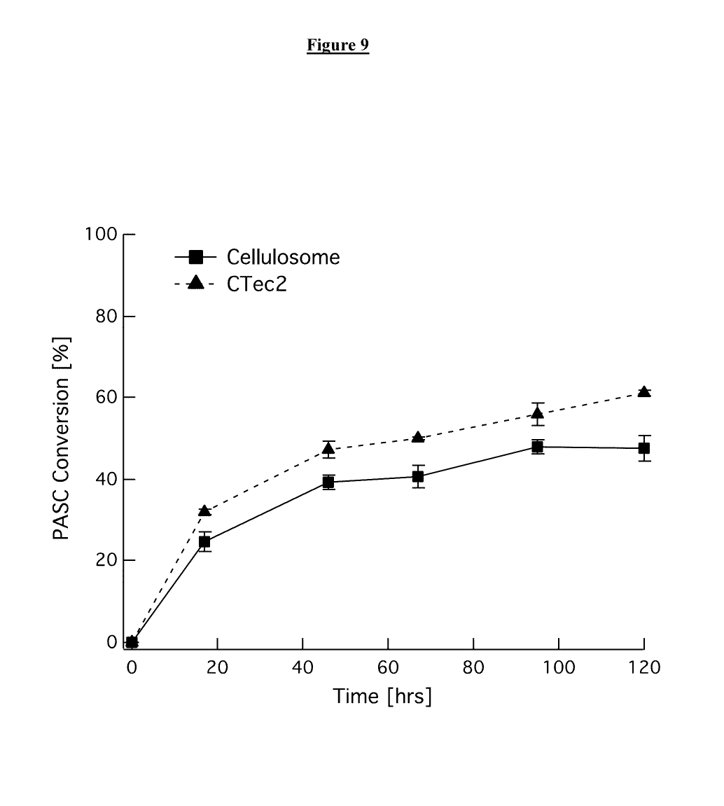

To examine the different mechanisms of the free and complexed enzyme systems on various model cellulose substrates, cellulosome activity (HMW cellulosomal fraction) was compared to a T. reesei enzyme preparation, desalted Cellic CTec2 (Novozymes). The performance of both enzyme systems with three model cellulose substrates was measured: Avicel PH-101, Whatman #1 filter paper, and phosphoric acid swollen cellulose (PASC). The first two substrates are primarily crystalline cellulose with varying degrees of polymerization (DP) whereas the lattermost is an amorphous, more accessible cellulose. For both systems, an enzyme loading of 5 mg of protein per gram of cellulose in a 1% (w/v) solids loading (10 mg/mL) was used. As shown in FIGS. 1A and 1B, cellulosomes are more efficient at converting crystalline cellulose exhibiting both low (Avicel PH-101, FIG. 1A) and high DP (Whatman #1 filter paper, FIG. 1B) than are the free enzymes. Using Avicel that is about 74% crystalline, three times as much Avicel is converted in 120 hours by the cellulosome in comparison with the free enzymes. Cellulosomes are also approximately three times more effective on Whatman #1 filter paper than CTec2 (FIG. 1B). In contrast, the cellulosomes are slightly less efficient than the free enzymes at hydrolyzing amorphous cellulose (phosphoric acid swollen cellulose (PASC); FIG. 9). These data suggest that cellulosomes are superior at degrading crystalline cellulose, whether with long or short DP.

### Example 8

### Hydrolysis of Pretreated Biomass by Free Enzymes and Cellulosomes

The ability of cellulosomes and free enzymes to mediate the hydrolysis of unpretreated and dilute-acid-pretreated biomass substrates was compared (FIGS. 1C and D and FIG. 10). As shown in FIG. 10B, 0.5 mm sieved, milled switchgrass is digested to 40% conversion by both of the cellulase systems after 48 hours, and the two enzyme systems do not continue to degrade biomass at an appreciable rate thereafter. This low level of glucan release likely reflects the limited enzyme accessibility of the cellulose and hemicellulose in unpretreated plant cell walls.

The activities of cellulosomes and free enzymes on switchgrass and poplar pretreated with dilute sulfuric acid are shown in FIGS. 1C and 1D, respectively. Cellulases were loaded at 20 mg per g of cellulose (FIG. 1C). In FIG. 1D, cellulosomes and CTec2 were compared at protein loadings of 5, 10, and 20 mg/g of cellulose. The gray “80 mg/g” curve represents digestion of pretreated poplar loaded with 80 mg/g cellulosomes. Biomass digestions were loaded with 2% solids.

Dilute sulfuric acid pretreatment both removes some lignin from the cell wall, a fraction of which condenses on cell wall surfaces upon cooling, and induces cell wall delamination. The switchgrass and poplar samples were milled, pretreated with dilute sulfuric acid, and extensively washed to remove soluble sugars, degradation products, and other soluble components. In contrast to the enzymatic performance on crystalline cellulose, where cellulosomes were found to be more effective, the free enzymes are faster at hydrolyzing pretreated biomass at the same protein loading (FIGS. 1C and D). The inactivity of the cellulosome is apparent after the first 24 hours of the reaction. To investigate if cellulosomes were limited by the lack of accessibility to the cellulose, the loading was varied from 5 to 20 mg/g and measured the conversion over 120 hours (FIG. 1D). The conversion increases with an increase of enzyme loading, which suggests that the reactive sites on the biomass surfaces are not fully saturated at the loadings tested. The conversion of pretreated switchgrass was also measured by supplementing the cellulosome with purified hemicellulases, which did not increase the glucan release as described in the SI (FIG. 10).

Additionally, the cellulosome loading was varied to determine if there was a point that would achieve the same conversion as the lowest loading of free enzymes. On pretreated poplar, a cellulosome loading of 80 mg per gram of glucan achieves the conversion level of CTec2 at 5 mg/g (FIG. 1D). Assuming a molecular weight average of 60 kDa for the free enzyme mixture and 1000 kDa for the cellulosome, the loadings of 80 mg cellulosome per g cellulose and 5 mg “free enzymes” per g cellulose are seen to be roughly equivalent on a molar basis, both amounting to loadings of approximately 0.8 micromoles protein per gram of cellulose.

### Example 9

### Cellulosomes Separation of Individual Cellulose Microfibrils

To investigate the morphological changes caused by the digestion of crystalline cellulose by either free enzymes or cellulosomes, additional digestions of Avicel were conducted using higher cellulosome loadings to produce substrate samples with approximately 65% of the cellulose removed. Avicel was digested to a cellulose conversion of ˜65% with free enzymes for 120 hours (A-D) and with cellulosomes for 24 hours (E-H).

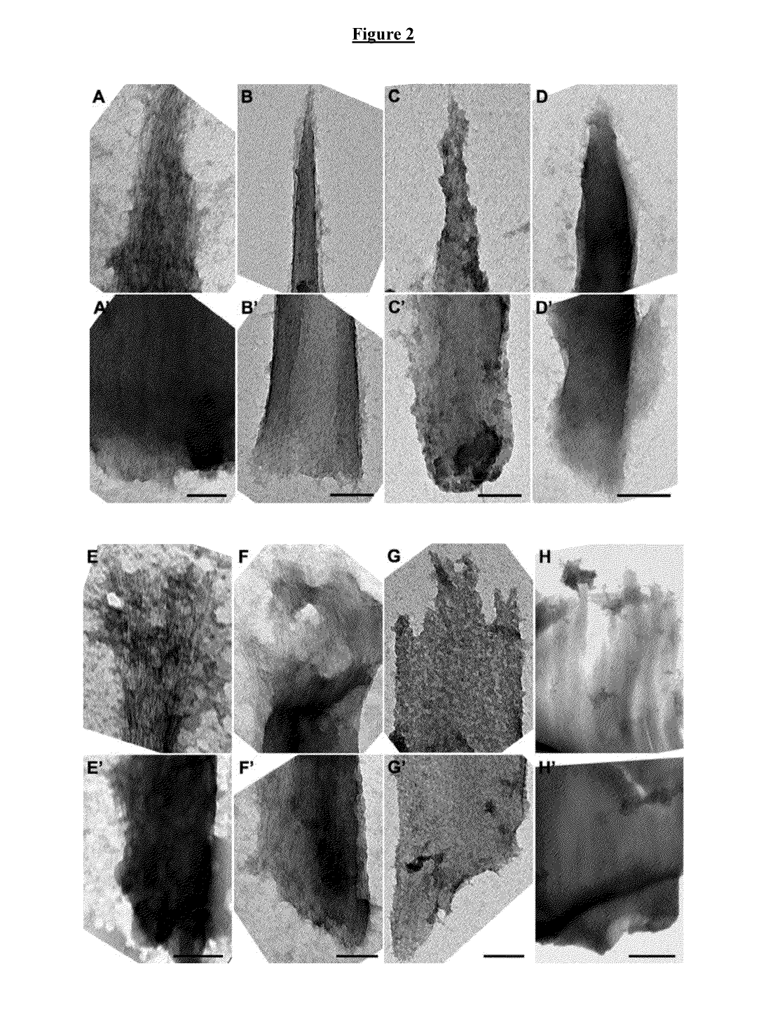

Digested Avicel particles were applied directly to a TEM grid, negatively stained, and imaged. Both samples displayed particles ranging in size from 3-580 μm² in cross sectional area with many of the particles still too thick to allow electron transmission without further sample preparation. The analysis focused on the smallest (0.5 μm⁻² μm wide), most electron-translucent particles within each sample, in which individual cellulose microfibrils could often be delineated within the bundles. Among this class of particles, there was a consistent pattern in the geometry of the particle ends. The particles digested with the free enzymes displayed one end that was tapered to a narrow point (FIG. 2A-D). The angle of the taper ranged from −6 to −12° measured between the particle edge and the long axis of the particle. The free enzymes appear to ablate the surface of cellulose microfibril bundles and work preferentially on one end only. The end of the particle opposite the tapered end was always either a blunt edge nearly perpendicular to the long axis of the particle or at an angle of about 60° (FIG. 2A′-D′). In contrast, the Avicel particles digested with cellulosomes do not display a tapered end, but instead exhibit an irregular and splayed end morphology (FIG. 2E-H). The angle of the splayed microfibrils ranged from 5 to 22° measured as a deflection away from the long axis of the particle. As in the free enzyme samples, in the cellulosome samples the end opposite the splayed end was either blunt, or in this case at an angle up to about 45° from the long axis of the particle (FIG. 2E′-H′). By measuring the perimeter of the particles in these two dimensional TEM micrographs as an approximation of the accessible surface area within 1 μm of the tapered or splayed end of the digested particles, an average 2-fold higher surface area in the splayed ends was calculated compared to tapered ends. This suggests that cellulosomes employ a mechanism distinct from the ablative mechanism of free cellulases, in that they separate individual cellulose microfibrils from crystalline cellulose particles for localized attack.

### Example 10

### Free and Cellulosomal Enzyme Localization on Pretreated Biomass

In addition to the imaging work on enzyme-digested Avicel, morphological changes in pretreated switchgrass digested by free enzymes or cellulosomes were also investigated. These samples were preserved by high-pressure freezing and freeze-substitution to keep structural details as close as possible to the structures actually present at the time the digestion was interrupted and to retain the antigenicity of enzyme epitopes for immuno-localization studies. The pretreated samples imaged before any exposure to cellulosomes or free enzymes were already extensively fractured and delaminated due to milling and pretreatment (FIG. 12).

To visualize changes in the pretreated biomass during enzymatic digestion, samples were collected from the digestion reactions at 4 and 24 hours. The samples were immuno-labeled to localize Cel7A enzymes (A, A′) or cellulosome scaffoldins (B, B′), which appear as black spots in the micrographs. Cel7A was concentrated within several μm of a cell lumen (CC, A) or cell corner (CC, A′), and after 24 hours, the enzymes penetrate into the secondary cell walls (2° CW). The cellulosome scaffoldin was found only near cell wall fractures (B, arrow) or very close to the cell wall surface (B′).

The imaging of these digested samples reveals an extreme variability within each sample, such that it is difficult to determine consistent morphological properties that distinguish the free enzyme digested samples from the cellulosome digested samples. However, the immuno-localization of enzyme penetration into the pretreated biomass provides insight. The distribution of Cel7A labeling in the free enzyme system shows that free enzymes have penetrated and dispersed into the secondary cell walls (FIG. 3A). Positive labeling for the cellulosome scaffoldin occurred only near fractures in the cell wall (FIG. 3B, arrow) or close to the cell wall surface (FIG. 3B′). These results suggest that accessibility to pretreated biomass is limited for the much larger, complexed enzymes.

### Example 11

### Synergy Between Free and Complexed Cellulases

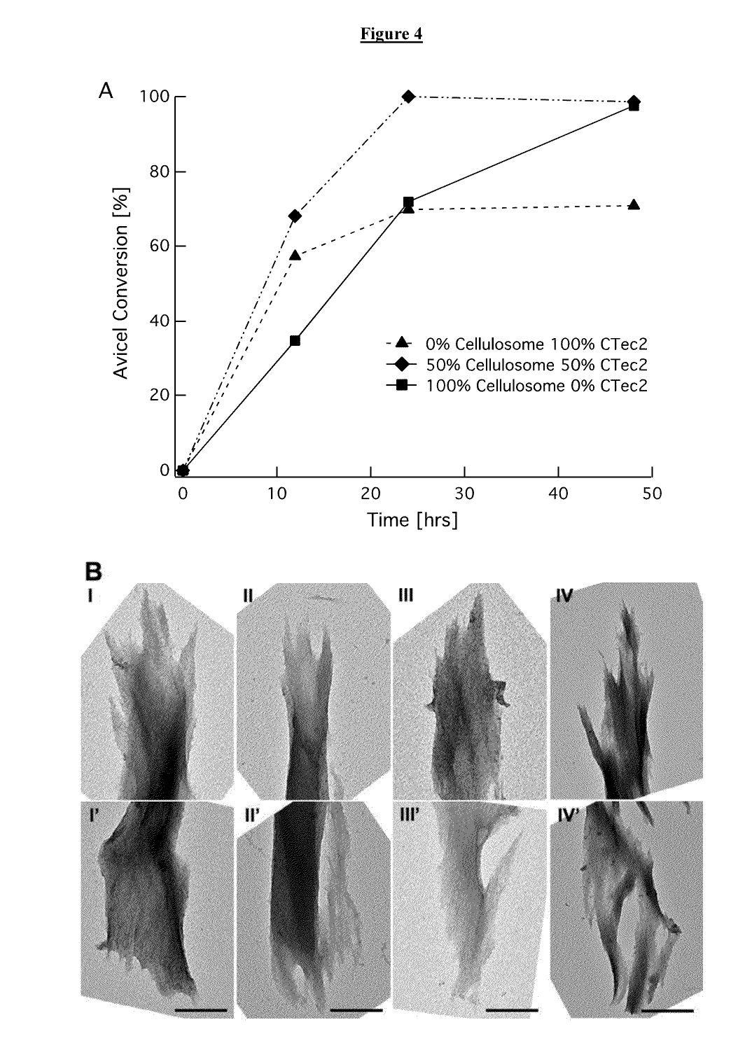

The results described above suggest that free cellulases and cellulosomes employ different physical mechanisms to break down recalcitrant polysaccharides. In particular, free enzymes appear to utilize an ablative mechanism, whereas cellulosomes appear able to separate individual cellulose microfibrils from one another for a localized increase in reactive cellulose surface area. To determine whether these two paradigms could be synergistic, a digestion of Avicel was conducted with a mixture of cellulosomes and free enzymes (FIG. 4). Cellulosomes and free enzymes were loaded at 10 mg/g in separate experiments, and a mixture of 5 mg/g of each was combined in enzymatic digestions of Avicel. Glucose and cellobiose release was measured every 12 hours by HPLC (A). Samples were taken at a conversion level of ˜55% for TEM image analysis (B I-IV) For all of the above reactions Avicel was loaded at 1% and the reaction was incubated at 50° C. in 25 mM NaAc, pH 5.0, 100 mM NaCl, 10 mM CaCl₂, 10 mM Cysteine, and 2 mM EDTA.

At 50° C., cellulosome initial activity was reduced compared to that of the free enzymes, because the cellulosomal enzymes were operating at sub-optimal temperature. However, the combination of cellulosomes and free enzymes exhibited the highest activity on Avicel (FIG. 4A). This suggests that the two mechanisms are complementary for the hydrolysis of clean, crystalline cellulose.

### Example 12

### Complementary Enzymatic Mechanisms Disrupt Cellulose Morphology

To investigate the mechanism of synergy between free and complexed enzymes, TEM imaging of Avicel that had been about 55% digested with a combination of CTec2 and cellulosomes was conducted (FIG. 4B I-IV). Interestingly, all of the cellulose particles imaged had a dramatically different morphology using the combined enzymes compared to either of the systems alone. Free enzymes sharpen the cellulose ends and cellulosomes cause splaying and surface area expansion. The combination of surface ablation and defibrillation is the result of the two enzyme systems working in a complementary relationship by exposing microcrystal ends to processive hydrolysis.

The Examples discussed above are provided for purposes of illustration and are not intended to be limiting. Still other embodiments and modifications are also contemplated.

While a number of exemplary aspects and embodiments have been discussed above, those of skill in the art will recognize certain modifications, permutations, additions and sub combinations thereof. It is therefore intended that the following appended claims and claims hereafter introduced are interpreted to include all such modifications, permutations, additions and sub-combinations as are within their true spirit and scope.

We claim:

1. A method for degrading cellulose or lignocellulosic biomass, comprising contacting a cellulose containing material or lignocellulosic biomass with an enzyme cocktail comprising at least one fungal cellulase and at least one high molecular weight (HMW) cellulosome complex.

2. The method of claim 1, wherein the at least one cellulosome complex is from a bacterium of the genus Clostridium.

3. The method of claim 2, wherein the bacterium is C. thermocellum.

4. The method of claim 1, wherein the at least one fungal cellulase comprises a Family 7 cellobiohydrolase.

5. The method of claim 4, wherein the Family 7 cellobiohydrolase is from a fungus of the genus Hypocrea.

6. The method of claim 5, wherein the fungus is H. jecorina.

7. The method of claim 6, wherein the Family 7 cellobiohydrolase is Cel7A.

8. The method of claim 4, wherein the enzyme cocktail further comprises a β-glucosidase.

9. The method of claim 4, wherein the enzyme cocktail further comprises at least one hemicellulase.

10. The method of claim 4, wherein the enzyme cocktail further comprises at least one oxidoreductase.

11. The method of claim 1, wherein the at least one fungal cellulase comprises CTec2.

12. The method of claim 1, wherein the contacting is carried out at a temperature of between 50-60° C.

13. The method of claim 1, wherein the contacting is carried out at a temperature of 50° C.

14. An enzyme cocktail comprising at least one HMW cellulosome complex and a Family 7 cellobiohydrolase.

15. The enzyme cocktail of claim 14, wherein the at least one HMW cellulosome complex is from C. thermocellum.

16. The enzyme cocktail of claim 14, wherein the Family 7 cellobiohydrolase is from Hypocrea jecorina.

17. The enzyme cocktail of claim 14, wherein the Family 7 cellobiohydrolase is Cel7A.

18. An enzyme cocktail comprising at least one HMW cellulosome complex and CTec2.

19. The enzyme cocktail of claim 18, wherein the at least one HMW cellulosome complex is from C. thermocellum.

20. A method for producing a biofuel from lignocellulosic biomass, comprising:
a) contacting the lignocellulosic biomass with the enzyme cocktail of claim 14; and
b) converting the sugars to a biofuel by fermentation.
